(19) United States
(12) Patent Application Publication
(10) Pub. No.: US 2007/0172520 A1
(43) Pub. Date: Jul. 26, 2007

(54) Immunotargeting of Nonionic Surfactant Vesicles

(75) Inventors: Michael VanAuker, Wesley Chapel, FL (US); Anna Plaas, Tampa, FL (US); Elizabeth Hood, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/561,639

(22) Filed: Nov. 20, 2006

Related U.S. Application Data
(60) Provisional application No. 60/738,173, filed on Nov. 18, 2005.

(51) Int. Cl. A61K 39/395; A61K 9/127

(52) U.S. Cl. 424/450; 424/144.1

(57) ABSTRACT

An immunoniosmes for targeted delivery of therapeutic agents to specific tissues in a host and methods of synthesis of those niosomes. An antibody molecule having specificity for a target antigen, such as a cell surface marker or other marker differentially expressed on a target cell, is covalently coupled to a functionalized membrane constituent. In a particular embodiment the functionalized membrane constituent is polyoxyethylene sorbitan monostearate functionalized with cyanuric chloride. The niosomes of this invention thus provide a composition that enhances internalization or retention of the bioactive agent of the niosome into the cytoplasm of the cells of the target tissue by providing a high degree of target specificity. Furthermore, the membrane vesicle enhances the life of the therapeutic agent by preventing its degradation in the extracellular environment, while exhibiting lower toxicity than can occur with some liposomes. The niosomes of the present invention are thus particularly useful as vehicles for the delivery of therapeutics to specific target cells.

### CROSS REFERENCE TO RELATED APPLICATIONS

This application claims priority to currently pending U.S. Provisional Patent Application No. 60/738,173, entitled, “Immunoniosome,” filed Nov. 18, 2005, the contents of which are herein incorporated by reference.

### FIELD OF INVENTION

This invention relates to the field of niosomes. More specifically, this invention relates to nonionic surfactant vesicles having enhanced targeting by the addition of a conjugated antibody on the surface of the niosomal vesicle.

### BACKGROUND OF THE INVENTION

Normal administration of drugs or therapeutic agents does not allow for concentrated accumulation of drug at diseased sites due to an essentially uniform distribution of drug throughout the body. In order to adequately treat affected sites using traditional systemic administration high dosages of drug must be delivered. This not only increases costs, but also can create toxic side effects as normal tissues and organs are needlessly exposed to pharmaceuticals (Torchilin (2000) European Journal of Pharmaceutical Sciences, 11, S81-S91.). Encapsulation of drugs for passive targeting, either by liposome (Chono et al. (2005) Journal of Drug Targeting, 13, 267-76; Park et al. (2004); Lasic and Martin, Stealth Liposomes, 1995), niosome (Lu et al. (2003) Drug Delivery, 10, 87-94; Balasubramaniam et al. (2002) Drug Development and Industrial Pharmacy, 28, 1181-1193; Gianasi et al. (1997) International Journal Of Pharmaceutics, 148, 139-148; Baillie et al., (1986) Journal Of Pharmacy And Pharmacology, 37, 863-868; Collins et al., (1993) Journal of Drug Targeting, 1, 133-42), or polymeric (Rapoport et al. (2003) Journal Of Controlled Release, 91, 85-95) media has shown increased retention time, decreased therapeutic dose, and reduced toxicity to unspecified tissues.

Drug targeting was originally conceived at the beginning of the 20th century by Paul Erlich who discovered antibodies and their role humoral immunity. He proposed the ‘magic bullet’ concept of using antibodies to send therapeutic agents to target cells (Torchilin (2000) European Journal Of Pharmaceutical Sciences, 11, S81-S91). Not until the development of monoclonal antibody (mAb) production could his ‘magic bullet’ concept be realized. In the 1970s the B cell melanoma was identified as producing a single type of antibody (Waldmann (2003) Nature Medicine, 9, 269-277) and the process of producing monoclonal antibodies was developed (Kohler and Milstein (1975) Nature, 256, 495-497). Later techniques were developed to humanize monoclonal antibodies for therapeutic uses (Riechmann et al., 1988 Nature, 332, 323-327).

Niosomes are self assembled vesicles composed primarily of synthetic surfactants and cholesterol (Baillie et al. (1985) Journal Of Pharmacy And Pharmacology, 37, 863-868; Torchilin (2000) European Journal Of Pharmaceutical Sciences, 11, S81-S91). They are analogous in structure to the more widely studied liposomes formed from biologically derived phospholipids. Vesicular drug delivery has been studied widely as a means to increase efficacy and reduce systemic toxicity in tumor targeting and cancer therapies (Xiong et al. (2005) Pharmaceutical Research, 22, 933-939; Uchegbu et al. (1996) Advances In Colloid And Interface Science, 58, 1-55; Sapra and Allen (2003) Progress In Lipid Research, 42, 439-462; Fonseca et al. (2005) European Journal Of Pharmaceutics And Biopharmaceutics, 59, 359-366). Echogenic liposomes have been developed for cardiovascular applications that can perform targeted imaging of atheroma (Dayton and Ferrara (2002) Journal Of Magnetic Resonance Imaging, 16, 362-377; Hamilton et al. (2004) Journal of the American College of Cardiology, 43(3): 453-60) and deliver drugs to the site of plaque development (Tiukinhoy et al. (2004) Investigative Radiology, 39, 104-110). Liposomes have been used to deliver fibrinolytics, such as streptokinase, and t-PA, and prostaglandin E1 (Feld et al. (1994) Journal Of The American College Of Cardiology, 24, 1382-1390; Heeremans et al. (1995) Thrombosis And Haemostasis, 73, 488-494; Nguyen et al. (1989) Proceedings Of The Society For Experimental Biology And Medicine, 192, 261-269).

Niosomes behave similarly to liposomes in vivo by prolonging circulation time of the encapsulated drug and altering chemical distribution within the body (Baillie et al., Journal Of Pharmacy And Pharmacology, 38, 502-505, 1986; Azmin et al., Journal Of Pharmacy And Pharmacology, 37, 237-242, 1985; Ruckmani et al., Drug Development And Industrial Pharmacy, 26, 217-222, 2000). However, niosomes have advantages over liposomes as drug carriers, including greater chemical stability, lower cost, easier storage and handling, and are less likely than liposomes to become toxic (Uchegbu and Florence, Advances In Colloid And Interface Science, 58, 1-55, 1995). Niosomal encapsulation reduces toxicity of drugs in many different applications and therapies. Niosomal drug delivery has been studied using various methods of administration (Blazek-Weish and Rhodes, AAPSpharmSci [electronic resource], 3, E1, 2001) including intramuscular (Arunothayanun et al., Journal Of Pharmaceutical Sciences, 88, 34-38, 1999), intravenous (Pillai and Salim, International Journal Of Pharmaceutics, 193, 123-127, 1999; Namdeo and Jam, Journal Of Microencapsulation, 16, 73 1-740, 1999), peroral (Rentel et al., International Journal Of Pharmaceutics, 186, 161-167, 1999), and transdermal (Uchegbu et al., Pharmaceutical Research, 12, 1019, 1995; Yoshioka et al., International Journal Of Pharmaceutics, 105, 1-6, 1994). Nebulized surfactants entrapping all-transretinoic acid (ATRA) were delivered as an inhaled aerosol reducing the drug toxicity and altering the pharmacokinetics (Desai and Finlay, International Journal Of Pharmaceutics, 241, 311-317, 2002). In addition, as drug delivery vesicles, niosomes have been shown to enhance absorption of some drugs across cell membranes (Lasic, Liposomes: from physics to applications/D. D. Lasic, 1993), to localize in targeted organs (Jam and Vyas, 1995; Namdeo and Jam, Journal Of Microencapsulation, 16, 73 1-740, 1999) and tissues (Baillie et al., Journal Of Pharmacy And Pharmacology, 38, 502-505, 1986; Azmin et al., Journal Of Pharmacy And Pharmacology, 37, 237-242, 1985), and to elude the reticuloendothelial system (Gopinath et al., Arzneimittel-Forschung-Drug Research, 51, 924-930, 2001).

Active drug targeting is generally described as the use of a vector molecule with a high specific affinity toward the affected tissues bound to a drug or drug carrier (Torchilin, European Journal Of Pharmaceutical Sciences, 11, S81-S91, 2000). The use of monoclonal antibodies or antibody fragments bound to drug carriers using differing carriers (Paukner et al., Molecular Therapy, 11, 215-223, 2005; Dinauer et al., Biomaterials, 26, 5898, 2005; Balthasar et al., Biomaterials, 26, 2723, 2005; Sapra and Allen, Progress In Lipid Research, 42, 439-462, 2003; Torchilin, Journal Of Controlled Release, 73, 137-172, 2001), and immunoconjugates (Volkel et al., Biochimica Et Biophysica Acta-Biomembranes, 1663, 158-166, 2004; Murciano et al., Blood, 101, 3 977-3984, 2003; Park et al., Journal Of Controlled Release, 74, 95-113, 2001) has been explored for varied medical applications (Dinauer et al., Biomaterials, 26, 5898, 2005; Sapra and Allen, Progress In Lipid Research, 42, 439-462, 2003; Gaidamakova et al., Journal Of Controlled Release, 74, 341-347, 2001; Mastrobattista et al., Biochimica Et Biophysica Acta-Biomembranes, 1419, 353-363, 1999; Torchilin, Advanced Drug Delivery Reviews, 17, 75-101, 1995). Liposomal immunotargeting has been used extensively for cancer and cardiovascular applications. Antibody-vesicle conjugation chemistries are varied but there are similar physical configurations that result in increased efficacy of antigen binding when the ligand is attached distal to the vesicle surface. This increases rotational freedom of the targeting moiety and decreases hindrance by the polyethelyne glycol at the surface of a ‘stealthy’ liposome (Zalipsky et al., Liposomes, Pt D, 2004). The addition of polyethylene glycol to a liposome to elude the reticuloendothelial system (RES) is well documented (Lasic and Martin, Stealth Liposomes, 1995; Uster et al., Febs Letters, 386, 243-246, 1996). Attachment of ligand distal to the vesicle on a PEG terminus was found to have increased binding to target cells compared to attachment on the surface (Hansen et al., Biochimica Et Biophysica Acta-Biomembranes, 1239, 133-144, 1995; Ishida et al., Febs Letters, 460, 129-133, 1999). Development of a PEG-PE end group functionalization allows for attachment of antibodies without prior derivatization of antibodies (Bendas et al., International Journal Of Pharmaceutics, 181, 79-93, 1999). Active targeting of niosomes was shown using glucose targeting with the inclusion of a glucose-palmitoyl glycol chitosan conjugate in a sorbitan monostearate niosome (Dufes et al., International Journal Of Pharmaceutics, 285, 77, 2004). Improved tumor targeting was shown using niosomes with PEG-glucose conjugates using a paramagnetic agent encapsulant (Luciani et al., Radiology, 231, 135-42, 2004).

Inflammatory processes play a role in vascular disease, rheumatoid and osteoarthritis, chronic obstructive pulmonary disease, and inflammatory bowel disease, lupus, among others. The inflammatory process is characterized by accumulation of inflammatory cells, leukocytes and macrophages, that perpetuate the process and contribute to tissue destruction. Inflammatory cells are recruited by cellular adhesion molecules (CAMs), which are glycoproteins expressed by the endothelium. CAMs mediate blood cell-endothelial cell interactions common to all segments of the vasculature under physiological or pathological conditions (Guray et al., International Journal of Cardiology, 96, 235, 2004). Increased development of atherosclerosis in rheumatoid arthritis patients without the traditional risk factors point to common mechanisms and the systemic implications of inflammatory pathologies (Hürlimann et al., Herz, 29, 760-8, 2004). In early atherosclerosis CAMs expressed on the vascular endothelium and on circulating leukocytes recruit inflammatory cells and facilitate their transport across the endothelium. Interruption of the inflammatory process has been studied using CD44 blocked by antibody IM7 (anti-CD44) (Gee et al., Archivum Immunologiae Et Therapiae Experimentalis, 52, 13-26, 2004). Expression of CD44 and its variants was augmented when exposed to pro-inflammatory cytokines within human atheroma, implicating CD44 expression with the pathogenesis of arterial diseases (Krettek et al., Am J Pathol, 165, 1571-1581, 2004). CD44 was further implicated in the progression of atherosclerosis. Atherosclerotic prone ApoE-deficient mice bred with CD44-null mice showed a 50-70% reduction in aortic lesions compared to CD44 heterozygous and wild type mice (Cuff et al., I Clin. Invest., 108, 1031-1040, 2001). These results suggest that CD44 promotes atherosclerosis by both mediating inflammatory cell recruitment to atherosclerotic lesions and by altering smooth muscle function (Cuff et al., I Clin. Invest., 108, 1031-1040, 2001). The primary ligand of CD44 is hyaluronan (HA), a principal glycosaminoglycan of the extracellular matrix. HA was shown, in a low molecular weight form, to stimulate vascular cellular adhesion molecule (VCAM-1) and proliferation of smooth muscle cells (SMC), whereas high molecular weight forms of HA inhibit SMC proliferation. Thus, manipulating the immunochemistry of pathogenic inflammatory cells and targeting ligands specific to reducing inflammatory response or disrupting an inflammatory cascade while concomitantly providing pharmaceutical therapeutics could prove valuable in combating inflammatory diseases.

### SUMMARY OF INVENTION

The present invention provides immunoniosmes for targeted delivery of therapeutic agents to specific tissues in a host and methods of synthesis of those niosomes. An antibody molecule having specificity for a target antigen, such as a cell surface marker or other molecule capable of recognition by an antibody and differentially expressed on a target cell, is covalently coupled to a functionalized membrane constituent. In a particular embodiment the functionalized membrane constituent is polyoxyethylene sorbitan monostearate functionalized with cyanuric chloride. The niosomes of this invention thus provide a composition that enhances internalization or retention of the bioactive agent of the niosome into the cytoplasm of the cells of the target tissue by providing a high degree of target specificity. Furthermore, the membrane vesicle enhances the life of the therapeutic agent by preventing its degradation in the extracellular environment, while exhibiting lower toxicity than can occur with some liposomes. The niosomes of the present invention are thus particularly useful as vehicles for the delivery of therapeutics to specific target cells.

In a first aspect the present invention provides a niosome for targeted delivery of a bioactive agent. The niosome includes a niosomal membrane comprising polyoxyethylene sorbitan monostearate and anan antibody or fragment thereof having specific affinity for a cell surface antigen wherein the antibody is covalently coupled to the polyoxyethylene sorbitan monostearate. The antibody or fragment thereof can be a anti-CD44 antibody, including its fragment. The niosome will advantageously include a bioactive agent such as an anti-inflammatory agent. In further advantageous embodiments the niosomal membrane further comprises sorbitan monostearate. The molar ratio of polyoxyethylene sorbitan monostearate to sorbitan monostearate can be about 1:5 to about 1:20. More particularly, the molar ratio of polyoxyethylene sorbitan monostearate to sorbitan monostearate is about 1:10.

In a second aspect the present invention provides a delivery reagent for the targeted delivery of a bioactive agent. The delivery agent includes a niosomal membrane comprising a nonionic surfactant molecule, an antibody or fragment thereof having specific affinity for an antigen and a cyanuric chloride linker. The cyanuric chloride linker couples the antibody or fragment thereof to the nonionic surfactant molecule. The antibody or fragment thereof can an anti-CD44 antibody. The delivery agent will advantageously include a bioactive agent such as an anti-inflammatory agent. In further advantageous embodiments the niosomal membrane further comprises sorbitan monostearate. The molar ratio of polyoxyethylene sorbitan monostearate to sorbitan monostearate can be about 1:5 to about 1:20. More particularly, the molar ratio of polyoxyethylene sorbitan monostearate to sorbitan monostearate is about 1:10.

In a third aspect the present invention provides a niosome for targeted delivery of an agent. The niosme includes a niosomal membrane comprising polyoxyethylene sorbitan monostearate and an antibody or fragment thereof having specific affinity for a cell surface antigen wherein the antibody is covalently coupled to the polyoxyethylene sorbitan monostearate.

The present invention further provides a method of preparing an immuno-conjugated niosome. The method includes the steps of providing a nonionic surfactant molecule comprising a terminal hydroxyl group on a chain of the molecule, functionalizing the hydroxyl group by addition of cyanuric chloride, constituting niosomal vesicles comprising the functionalized nonionic surfactant molecule and reacting the functionalized nonionic surfactant molecule with an antibody or fragment thereof to form a nonionic surfactant molecule covalently coupled to an antibody. The constituted niosomal vesicle can advantageously include a bioactive agent thus providing a delivery agent for the targeted delivery of the bioactive compound. In certain embodiments the nonionic surfactant molecule comprising a terminal hydroxyl group can be polyoxyethylene sorbitan monostearate. In further advantageous embodiments the niosomal membrane further comprises sorbitan monostearate. The molar ratio of polyoxyethylene sorbitan monostearate to sorbitan monostearate can be about 1:5 to about 1:20. More particularly, the molar ratio of polyoxyethylene sorbitan monostearate to sorbitan monostearate is about 1:10.

### BRIEF DESCRIPTION OF THE DRAWINGS

For a fuller understanding of the invention, reference should be made to the following detailed description, taken in connection with the accompanying drawings, in which:

FIG. 1 is an illustration representing a cross-section of a surfactant functionalized niosome membrane conjugated to monoclonal IgG antibodies. Components on right: top; cyanuric chloride functionalized Tween 61, top middle; cholesterol molecule. Bottom, middle; Span 61 and bottom; monoclonal IgG antibody.

FIG. 2 is an illustration of the structure of polyoxyethylene sorbitan monostearate (Tween 61) molecule.

FIG. 3 shows the mechanism of cyanuric chloride binding to Tween 61.

FIG. 4 shows the UV absorbance elution profile of a 10% Tween GEC purification. The v₀ peak represents 280 nm absorbance of CF entrapped in the niosomes.

FIG. 5 shows the mechanism of cyanuric chloride linking an IgG antibody to the functionalized Tween 61 molecule incorporated in the niosome membrane.

FIG. 6 is a graph illustrating the stability of niosomes, as defined as the fraction of dye remaining entrapped, over time. “% Tween” is the percentage of total surfactant that is Tween 61. The remaining surfactant is Span 60.

FIG. 7 shows the absorption of Alexa Fluor 488 tagged IgG conjugated to niosomes is evident in the v₀ elution peak.

FIG. 8 is a fluorescent micrograph of niosomes conjugated to fluorescently tagged IgG antibodies.

FIG. 9 is a series of micrographs demonstrating the binding of immunoniosomes to target antigens. The upper slides A, C, & E show contrast micrographs and the lower slides B, D, & E are the corresponding fluorescent micrographs of those above them. A & B show SL cells incubated with IM7-tagged niosomes. C&D. SL cells pre-incubated with free IM7. E&F. SL cells incubated with untagged niosomes.

FIG. 10 shows SL cells stained for CD44 with nuclear counterstaining. Arrows indicate CD44 expressed on cell processes.

FIG. 11 shows SL cells incubated with IM7 conjugated niosomes. Fluorescent image is an overlay of green (FITC) and blue (DAPI) fluorescence and contrast images showing attachment of IM7-tagged niosomes.

FIG. 12 shows that IM7-conjugated immuniosomes adhere to the surface of CD44-expressing cells; (left) overlay of contrast micrograph of SL cells with the fluorescent DAPI (middle) stain of cell nuclei, and FITC (right) immunoniosome images.

### DETAILED DESCRIPTION OF THE PREFERRED EMBODIMENT

The present invention provides immunoniosmes for selective delivery of therapeutic agents to specific tissues in a host and methods of synthesis of those niosomes. An antibody molecule having specificity for a target antigen, such as a cell surface marker or other marker differentially expressed on a target cell, is covalently coupled to a functionalized membrane constituent. In a particular embodiment the functionalized membrane constituent is polyoxyethylene sorbitan monostearate functionalized with cyanuric chloride niosome vesicle

The niosomes of this invention thus employ a composition that optimizes internalization or retention of the bioactive agent of the niosome into the cytoplasm of the cells of the target tissue. The phrase “optimizes internalization” or “optimal internalization” is used to refer to the delivery of niosome contents such that it achieves enhanced delivery to the cytoplasm of the target cell and therefore enhanced therapeutic effect. The immunoniosomes of this invention optimize delivery of the therapeutic agent by providing a high degree of target specificity. Without being bound by a particular theory, one explanation for the observed increase in delivery is that the antibody presented on the surface of the niosome results in the effective internalization of the niosome itself (carrying therapeutic agent) thereby avoiding considerable loss of the therapeutic agent in solution or degradation of the therapeutic in the endosomic/lysosomic pathway. The niosomes of the present invention are thus particularly useful as vehicles for the delivery of therapeutics to specific target cells.

Terminology

An “antigen” is any substance that can bind to a specific antibody. Proteins, carbohydrates, nucleic acids and other molecules are all potential antigens.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

An “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies may exist as intact immunoglobulins or as a number of well characterized fragments produced by digestion with various peptidases. In particular, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially an Fab with part of the hinge region (see, Fundamental Immunology, W. E. Paul, ed., Raven Press, N.Y. (1993) for more antibody fragment terminology). While the Fab′ domain is defined in terms of the digestion of an intact antibody, one of skill will appreciate that such Fab′ fragments may be synthesized de novo either chemically or by utilizing recombinant DNA methodology.

The antibody is selected to specifically bind to a molecule or marker characteristic of the surface of the cells to which it is desired to deliver the contents of the niosome. A molecule is characteristic of cell, tissue, or physiological state when that molecule is typically found in association with that cell type or alternatively in a multiplicity of cell types all expressing a particular physiological condition (e.g., transformation). A specific characteristic marker is preferably found on the surfaces of cells of a particular tissue or cell type or on the surfaces of tissues or cells expressing a particular physiological condition and on no other tissue or cell type in the organism. One of skill will recognize however, that such a level of specificity of the marker is often not required. For example a characteristic cell surface marker will show sufficient tissue specificity if the only non-target tissues are not accessible to the niosome. Alternatively, effective specificity may be achieved by overexpression of the marker in the target tissue as compared to other tissues. This will result in preferential uptake by the target tissue leading to effective tissue specificity. Thus for example, many cancers are characterized by the overexpression of cell surface markers such as the HER2 (c-erbB-2, neu) proto-oncogene encoded receptor in the case of breast cancer.

One of skill will recognize that there are numerous cell surface markers that provide good characteristic markers for niosomes depending on the particular tissue it is desired to target. These cell surface markers include, but are not limited to carbohydrates, proteins, glycoproteins, MHC complexes, and receptor proteins such as HER, CD4 and CD8 receptor proteins as well as other growth factor receptor proteins. Growth factor receptors are cell surface receptors that specifically bind growth factors and thereby mediate a cellular response characteristic of the particular growth factor. The term “growth factor”, as used herein, refers to a protein or polypeptide ligand that activates or stimulates cell division or differentiation or stimulates biological response like motility or secretion of proteins. Growth factors are well known to those of skill in the art and include, but are not limited to, platelet-derived growth factor (PDGF), epidermal growth factor (EGF), insulin-like growth factor (IGF), transforming growth factor β (TGF-β), fibroblast growth factors (FGF), interleukin 2 (IL2), nerve growth factor (NGF), interleukin 3 (IL3), interleukin 4 (IL4), interleukin 1 (IL1), interleukin 6 (IL6), interleukin 7 (IL7), granulocyte/macrophage colony-stimulating factor (GM-CSF), granulocyte colony-stimulating factor (G-CSF), macrophage colony-stimulating factor (M-CSF), erythropoietin and the like. One of skill in the art recognizes that the term growth factor as used herein generally includes cytokines and colony stimulating factors.

It will be appreciated that the characteristic marker need not be a naturally occurring marker, but rather may be introduced to the particular target cell. This may be accomplished by directly tagging a cell or tissue with a particular marker (e.g., by directly injecting the particular target tissue with a marker, or alternatively, by administering to the entire organism a marker that is selectively incorporated by the target tissue. In one embodiment, the marker may be a gene product that is encoded by a nucleic acid in an expression cassette. The marker gene may be under the control of a promoter that is active only in the particular target cells. Thus introduction of a vector containing the expression cassette will result in expression of the marker in only the particular target cells. One of skill in the art will recognize that there are numerous approaches utilizing recombinant DNA methodology to introduce characteristic markers into target cells.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The terms “ligand” or “targeting moiety”, as used herein, refer generally to all molecules capable of specifically binding to a particular target molecule and forming a bound complex as described above. Thus the ligand and its corresponding target molecule form a specific binding pair. Examples include, but are not limited to antibodies, lymphokines, cytokines, receptor proteins such as CD4 and CD9, solubilized receptor proteins such as soluble CD4, hormones, growth factors, and the like which specifically bind desired target cells, and nucleic acids which bind corresponding nucleic acids through base pair complementarity. Particularly preferred targeting moieties include antibodies and antibody fragments.

The term “immunoniosome” refers to a niosome bearing an antibody or antibody fragment that acts as a targeting moiety enabling the liposome to specifically bind to a particular “target” molecule that may exist in solution or may be bound to the surface of a cell.

Immunoniosome Contents

Active agents suitable for use in the present invention include therapeutic drugs and pharmacologically active agents, nutritional molecules, cosmetic agents, diagnostic agents and contrast agents for imaging. As used herein, active agent includes pharmacologically acceptable salts of active agents. Suitable therapeutic agents include, for example, antineoplastics, antitumor agents, antibiotics, antifungals, anti-inflammatory agents, immunosuppressive agents, anti-infective agents, antivirals, anthelminthic, and antiparasitic compounds.

In treating tumors or neoplastic growths, suitable compounds may include anthracycline antibiotics (such as doxorubicin, daunorubicin, carinomycin, Nacetyladriamycin, rubidazone, 5-imidodaunomycin, N30 acetyldaunomycin, and epirubicin) and plant alkaloids (such as vincristine, vinblastine, etoposide, ellipticine and camptothecin). Other suitable agents include paclitaxel (TAXOL®; a diterpenes isolated from the bark of the yew tree and representative of a new class of therapeutic agents having a taxane ring structure) and docetaxol (taxotere); mitotane, cisplatin, and phenesterine.

Anti-inflammatory therapeutic agents suitable for use in the present invention include steroids and non-steroidal anti-inflammatory compounds, such as prednisone, methyl-prednisolone, paramethazone, 11-fludrocortisol, triamciniolone, betamethasone and dexamethasone, ibuprofen, piroxicam, beclomethasone; methotrexate, azaribine, etretinate, anthralin, psoralins; salicylates such as aspirin; and immunosuppresant agents such as cyclosporine. Anti-inflammatory corticosteroids and the anti-inflammatory and immunosuppressive agent cyclosporine are suited for use in the present invention. Antineoplastic agents can also be used.

Additional pharmacological agents suitable for use in niosomes of the present invention include anesthetics (such as methoxyflurane, isoflurane, enflurane, halothane, benzocaine, lidocane, bupivocane, and ropivicane); antiulceratives (such as cimetidine); antiseizure medications such as barbituates; azothioprine (an immunosuppressant and antirheumatic agent); and muscle relaxants (such as dantrolene and diazepam).

Imaging agents suitable for use in the present niosome preparations include ultrasound contrast agents, radiocontrast agents (such as radioisotopes or compounds containing radioisotopes, including iodo-octanes, halocarbons, and renograf in), or magnetic contrast agents (such as paramagnetic compounds).

Nutritional agents suitable for incorporation into niosomes of the present invention include flavoring compounds (e.g., citral, xylitol), amino acids, sugars, proteins, carbohydrates, vitamins and fat. Combinations of nutritional agents are also suitable.

The above active agents may be used in the various niosome embodiments described, but not limited to, those described hereinabove. Additionally, it should be emphasized that the niosomes may comprise a single pharmacologically active agent (e.g., at least one active agent) or multiple active agents, depending on the intentions of the administrator. Embodiments utilizing multiple active agents within the same niosome or in two separate niosome formulations administered together, may provide enhanced efficacy due to syngergistic behavior by the agents.

The invention is described below in examples which are intended to further describe the invention without limitation to its scope.

Non ionic surfactant vesicles (niosomes) composed of sorbitan monostearate (Span 60), polyoxyethylene sorbitan monostearate (Tween 61), cholesterol, and dicetyl phosphate were conjugated with a purified monoclonal antibody to CD44 (IM7) through a cyanuric chloride (CC) linkage on the polyoxyethylene group of the Tween 61 molecule. Inclusion of small amounts of Tween 61 within the surfactant component of niosomes formed using thin film hydration techniques and sonication did not hamper vesicle stability as compared to Span 60 niosomes. Conjugation was verified by UV absorbance of fluorescently tagged IM7 in non fluorescing niosomes and fluorescent micrographs. The immuno-niosomes were incubated with synovial lining cells expressing CD44. Attachment of niosomes was evident and showed selectivity and specificity compared to controls. Cell binding density was determined to be 2.29+/−0.26 immunoniosomes per cell at a cell density of 2.20×10⁵ cells/cm². These findings show that the resulting immunoniosomes may an effective method for targeted drug delivery.

### EXAMPLE 1

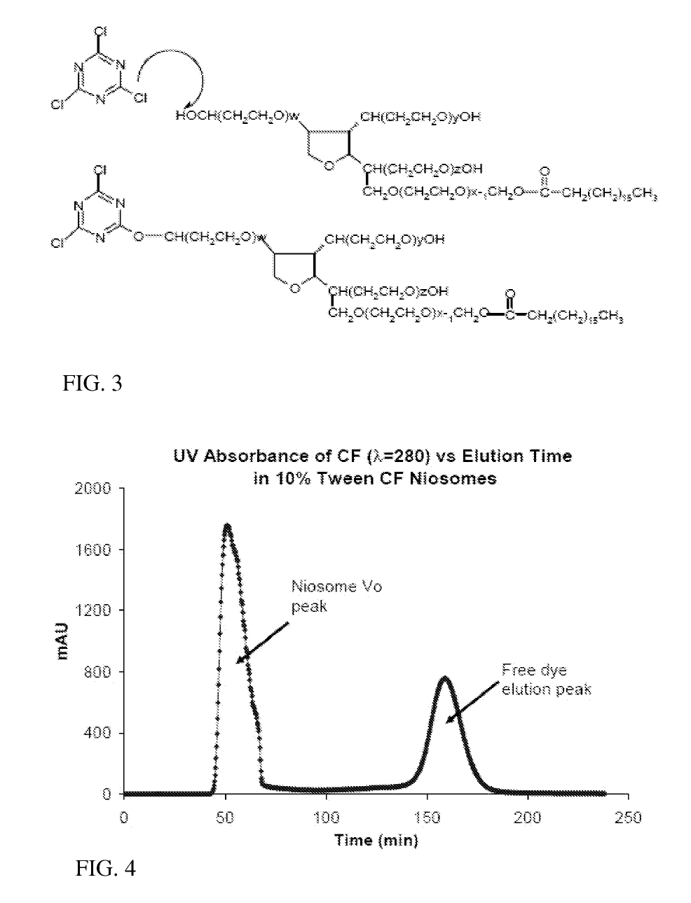

The overall scheme in synthesizing an immunoniosome drug carrier was to first chemically modify a surfactant component, polyoxyethylene sorbitan monostearate (Tween 61), to create a linker, and then to incorporate the surfactant-linker within the niosome membrane, and finally to incubate the functionalized niosomes with monoclonal antibodies to achieve conjugation. Niosomes were synthesized by classical thin-film hydration methods (Yoshioka T, Stemberg B, Florence A T: Preparation And Properties Of Vesicles (Niosomes) Of Sorbitan Monoesters (Span-20, Span-40, Span-60 And Span-80) And A Sorbitan Triester (Span-85). International Journal Of Pharmaceutics 1994;105:1-6) with a mixture of biocompatible sorbitan ester surfactants and cholesterol using both agitation and sonication during the hydration phase. We conjugated the formed vesicles to IM7 antibodies through a novel polyoxyethylene sorbitan monostearate (Tween 61)-cyanuric chloride (CC) linker incorporated in the vesicle membrane. Tween 61 was functionalized prior to niosome synthesis by activation the hydroxyl groups on the ends of the polyethylene oxide (PEO) chains. In the presence of diisopropyl ethyl amine (DIPEA), Tween 61 and cyanuric chloride are incubated in a nitrogen environment. The cyanuric chloride undergoes nucleophilic substitution binding to the terminal hydroxyl group of a PEC chain on the Tween 61 molecule as shown in FIG. 3.

The molar ratio of Tween:CC:DIPEA was 1:0.8:2. The resulting functionalized TweenCC solution added to the surfactants and lipids in chloroform prior to forming a thin film. Vesicles are composed of a 1.0:1.0:0.1 molar ratio of surfactant:cholesterol:DCP at a concentrations between 0.0144 and 0.144 M. Niosomes were separated from unencapsulated dye and unformed lipids using gel exclusion chromatography by passing vesicles through a Sephadex G50 column with a 0.01 M PBS mobile phase at a flow rate of 1.0 ml/min.

Entrapment of fluorescent dye used as a drug model was measured using both UV absorbance during the purification step, and fluorescence intensity of disrupted vesicle suspensions relative to a standard curve. Formation of vesicles was assessed by light and fluorescent microscopy. Mean particle size and distribution of formed vesicles was determined by light scattering and obscuration techniques.

Once formed, niosome solutions were adjusted to pH 8.8 and were incubated with monoclonal anti-CD44 IM7 antibodies. At pH 8.8 the binding of a terminal carboxyl group on the antibody is preferred over that of an amino group at the antigen binding terminus. The resulting ‘immunoniosomes’ bind selectively and specifically to CD44 antigen targets on synoviocytes, our initial cell model, at IgG concentrations far lower than advocated by traditional immunoliposome literature (Allen T M, Brandeis E, Hansen C B, Kao GY, Zalipsky 5: A New Strategy For Attachment Of Antibodies To Sterically Stabilized Liposomes Resulting In Efficient Targeting To Cancer-Cells. Biochimica Et Biophysica Acta-Biomembranes 1995; 1237:99-108.). Concentration of antibodies incubated was 5 μg protein/ml niosomes which is equivalent to 2.78 μg protein/μmol lipid. Antibody-niosome binding was evaluated by UV absorbance and fluorescence microscopy.

Once the conjugation was verified, binding of immunoniosomes to target antigen in a fixed cell model was assessed. Bovine synoviocytes were used in niosome incubation experiments. The cells were grown in Dulbecco's Modified Eagle Medium (DMEM) containing 10% fetal calf serum and subcultured at a concentration of 10⁵ cells per ml into 8 well microslides for niosome incubation. Cells were allowed to attach overnight. The media was removed, and the cells washed with PBS and fixed for 2 hours in Histochoice. The fixed cell layers were pre-incubated in 0.01 M PBS with 2% goat serum with or without soluble IM7 antibody for 1 hour at room temperature prior to incubation with immunoniosomes. Cells were rinsed with PBS and incubated for 1 hour at 37° C. with fluorescent niosomes or fluorescent niosomes derivatized with IM7. The cells were well rinsed to remove unbound niosomes and examined by fluorescent microscopy. The cell study's two controls included cells preincubated with IM7 to block binding sites, and cells incubated with non immono-tagged niosomes. Both phase contrast and fluorescent images of post incubated cells were captured with an Olympus 1X71 inverted fluorescent microscope and combined using DP-BSW image analysis software to overlay images. Original images were retained for digital analysis to quantify binding. Some cells incubated with immunoniosomes were also DAPI stained to visualize the cell nuclei and the resulting blue and green fluorescent images were combined with the light image.

To quantify the amount of adherence of fluorescent immunoniosomes to cells we developed an image analysis program using Matlab® version 7.1. The program prompts the user to import the fluorescent images representing the number of cells (blue DAPI image), and the number of immunoniosomes (green FITC images). From the micrograph scale bar the program creates a scale. The user is prompted to crop the total image. Each blue cell image is identified as an object, and then measured and counted based on the average area of all the objects. Very small objects are filtered out of the cell count, and very large objects (closely packed cell membranes) are divided by the mean area overall to determine the number of cells represented. The program computes a cell count and a cell density based on number per unit area. The FITC image is processed similarly; each individual object is identified, analyzed, and counted. The immunoniosome binding density is a simple ratio of number of green objects identified to the blue objects.

A contrast and fluorescence overlay micrograph of IM7 conjugated immunoniosomes shows adherence to the surfaces of CD44 expressed cells in the left image of FIG. 12. The image is the overlay of the contrast image with the DAPI (blue) image of the cell nuclei, and the FITC (green) image of the immunoniosomes. Results are for a 1 hour incubation of immunoniosomes at a concentration of approximately 3.0×10 #/ml, and 720 nm as measured by light scattering and obscuration at a limitation of particles measured at diameters greater than 500 nm. Niosome cell binding density was determined to be 2.29+/−0.26 immunoniosomes per cell at a cell density of 2.20×10 cells/cm2 with a standard error of the mean of less than 3% at an n=12 measurements taken per image. Controls did not show any non specific binding of immunoniosomes or any untargeted interactions of nonimmunoniosomes to cell surfaces.

These results confirm the capacity to develop monoclonal antibody conjugated niosomes targeted to specific cell receptors. Sorbitan monostearate based niosomes can be functionalized through inclusion of a cyanuric chloride derivatized polyoxyethylene monostearate to conjugate monoclonal IgG antibodies to the vesicle surfaces without requiring derivatization of the antibody. The resulting immunoniosome can bind to target antigens in fixed cells. In the fixed cell model targeting shows high selectivity and specificity. Further studies will be conducted to investigate the optimal particle concentration and antibody density.

Since the attachment of antibodies is independent of the type and generic to any IgG antibody, the system's therapeutic targeting is flexible and may include more than one targeting vector if desired. The anti-CD44 antibody IM7 was conjugated to sorbitan ester based niosomes via a cyanuric chloride linkage and targeted to fixed cells known to express CD44. Further exploration of the capacity of vesicle binding and subsequent uptake in endothelial cells of the immuno-niosomes will test the potential of the system to not only target inflammatory disease but also to deliver anti-inflammatory agents.

### EXAMPLE 2

The effect of the inclusion of a range of small molar percentages of Tween 61 in a Span 60 niosome on vesicle entrapment capacity and membrane stability was evaluated and measured by retention of entrapped dye over time at 4° C. in a PBS suspension. Although Tween 61 niosomes are reported to have a greater entrapment capacity (Manosroi et al., International Journal Of Pharmaceutics, 298, 13-25, 2005), we observed that niosomes whose surfactant component was entirely composed of Tween 61 lost three times more encapsulated dye relative to niosome formulations whose surfactant component was purely Span 60 under static conditions holding cholesterol and DCP molar ratios constant (FIG. 6). This figure also shows that the inclusion of up to 10% by mole of Tween 61 showed no statistical effect on retention.

### EXAMPLE 3

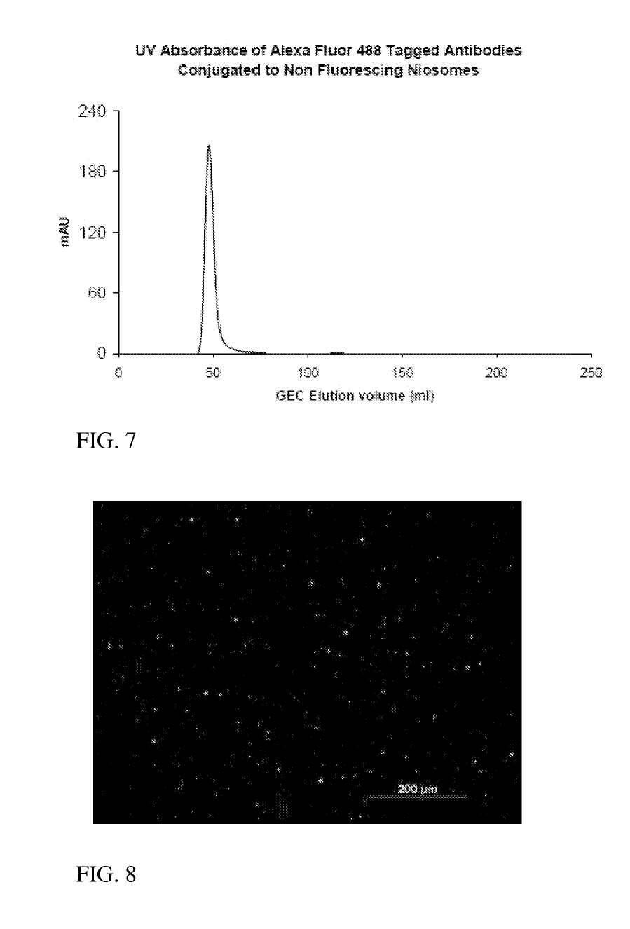

Successful attachment of Alexa Fluor tagged IgGs to PBS containing niosomes is demonstrated by the observance of UV absorbance at 280 nm at the GEC elution void volume in FIG. 7. A very slight signal at 120 ml indicates that few of the AF antibodies were left unbound since the protein and the bound dye would both contribute to the signal at that wavelength. Further demonstration of the conjugation of AF antibodies is seen in the fluorescent image in FIG. 8. The discrete fluorescent spheres appear to be of the same size and relative size distribution of niosomes. These two independent measures indicate successful antibody conjugation of the AF antibodies to non fluorescing niosomes.

### EXAMPLE 4

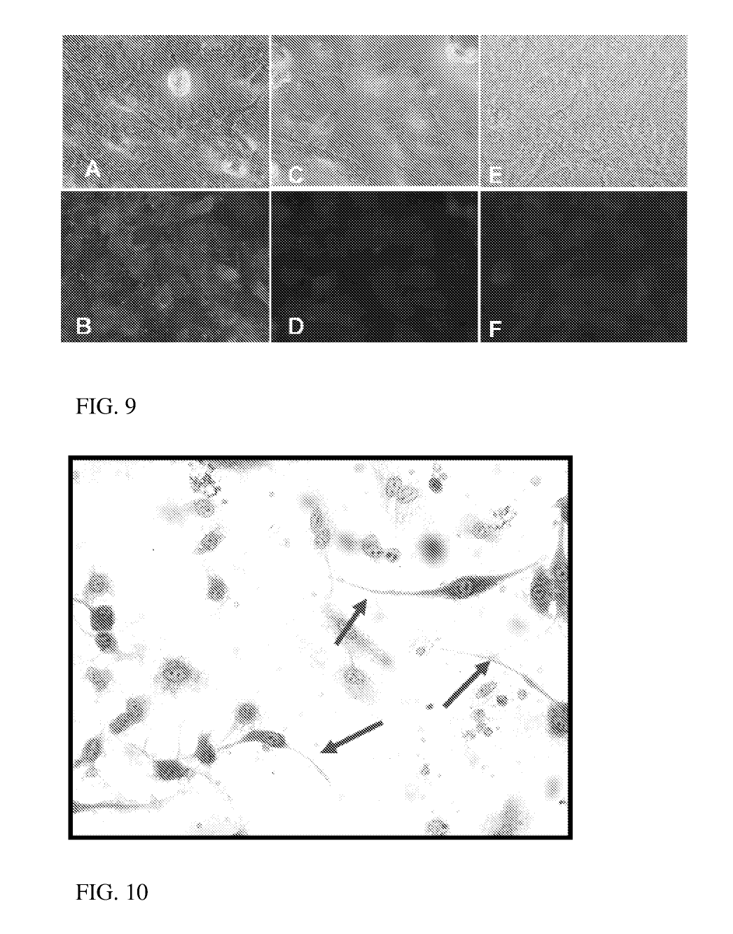

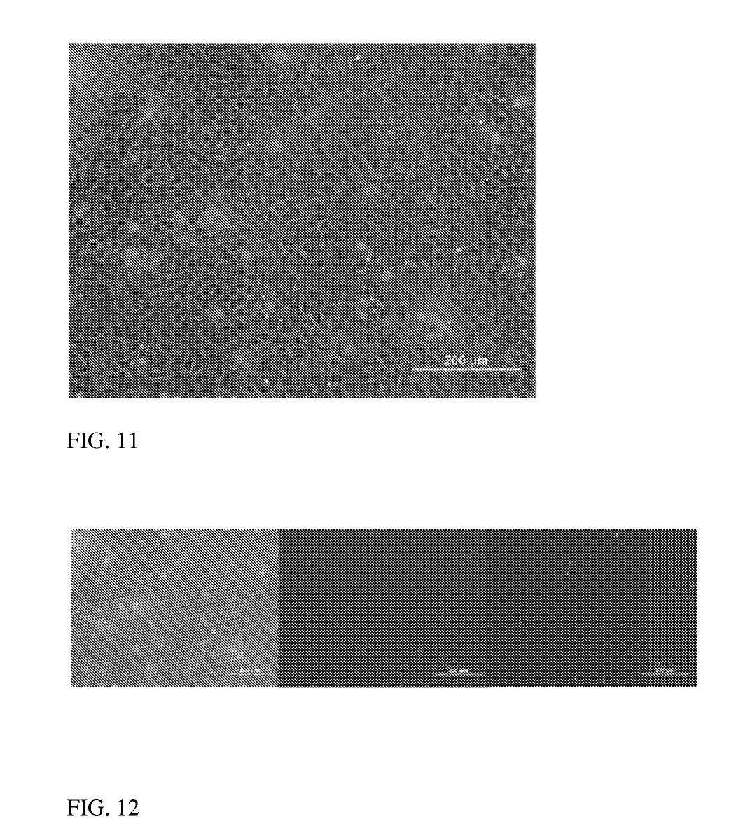

Binding of immunoniosomes to target antigen in a fixed cell model is confirmed as follows. The fluorescent and light micrographs demonstrate the specificity and selectivity of immunoniosome binding to target antigens (FIG. 9) The upper micrograph figures of cells (A, C, and E) correspond exactly to the fluorescent micrographs below them (B, D, and F). Parts A and B of FIG. 9 correspond to the cells incubated with IM7 tagged niosomes and show binding of the immunoniosomes evident by the bright spherical shapes attached at cell processes and cell membranes. Whereas the cells pre-incubated with free IM7, shown in C and D, do not show the small spherical attachments due to blocking of the targeted binding sites. This demonstrates the targeting selectivity of antibody antigen binding. In parts E and F, cells have been incubated with unconjugated niosomes. Additionally, the absence of binding in the cells incubated with untagged niosomes, parts E and F, demonstrates the specificity. In all of the fluorescence images some autofluorescence of cells is evident but clearly distinct from the brighter pointlike images of the fluorescent niosomes. In FIG. 10, CD44 expression at the cell processes and at the cell membranes shown using IHC staining techniques. Correspondingly binding of IM7 immunoniosomes in FIG. 11 is seen at cell processes and membranes.

The Tween-CC chloroform solution was found to be viable up to 6 months stored at −4 CC. Conjugation of antibodies to Tween.

These results confirm the capacity to develop monoclonal antibody conjugated niosomes targeted to specific cell receptors. Sorbitan monostearate based niosomes can be functionalized through inclusion of a cyanuric chloride derivatized polyoxyethylene monostearate to conjugate monoclonal IgG antibodies to the vesicle surfaces without requiring derivatization of the antibody. The coupling of antibodies to vesicles is efficient. The resulting ‘immunoniosome’ can bind to target antigens in fixed cells. In the fixed cell model targeting shows high selectivity and specificity. Further studies will be conducted to investigate uptake by inflamed endothelial cells in vitro and in vivo. Since the attachment of antibodies is independent of the type and generic to any IgG antibody, the system's therapeutic targeting is flexible and may include more than one targeting vector if desired.

In this report, the anti CD44 antibody IM7 was conjugated to sorbitan ester based niosomes via a cyanuric chloride linkage and targeted to fixed cells know to express CD44. Further exploration of the capacity of vesicle binding and subsequent uptake in endothelial cells of the immuno-niosomes will test the potential of the system to not only target inflammatory disease but also to deliver anti-inflammatory agents. Further in vitro studies will involve quantifying immunoniosome binding to endothelial cells and then assessing uptake into cells The implications for therapeutic treatment of inflammatory diseases is significant not only in the capacity to target chemical therapy to affected tissues but also by blocking receptors of inflammatory pathways and interrupting the perpetuating effect of the process (Pure and Cuff, Trends In Molecular Medicine, 7, 213-221, 2001).

### EXAMPLE 5

Inflammatory processes play a role in vascular disease. Expression of cellular adhesion molecules such as CD44 is upregulated in inflamed endothelium and therefore may promote atherosclerosis. This may provide a target for delivery of therapeutics specific to reducing or disrupting inflammation. Nonionic surfactant vesicles (niosomes) are potential stable, low cost drug delivery vehicles that have been shown in other contexts to be useful in lowering systemic doses of drug for efficacy. We addressed the hypothesis that niosomes could be synthesized that could specifically target the CD44 molecule.

Niosomes containing fluorescent green carboxyrhodamine dye were created using a combination of modified surfactants. Through a novel process we developed, we conjugated these to anti-CD44 (IM7) monoclonal antibodies to form “immunoniosomes”. These were incubated with bovine aortic endothelial cells (BAECs) activated with TNF-α. to express CD44. To verify and quantify binding, phase contrast and fluorescent images of post incubated cells were captured. Cells were DAPI blue stained to visualize the nuclei. The number of immuno-niosomes bound to cells was determined through a custom-made image analysis program. Binding density was defined as a ratio of the number of immuno-niosomes per cell nucleus. Controls included cell incubation with unconjugated niosomes and BAECs pre-incubated with free IM7 to block binding sites.

A subgroup of BAECs underwent immunohistochemical staining which verified expression of CD44 on the cell surface. Comparison of experiments with immunoniosomes versus controls revealed good selectivity and specificity. For a 1 hour incubation of immuno-niosomes at a concentration of 3.0×10⁷ #/ml and a cell density of 2.20×10⁵ cells/cm², binding density was 2.29+/−0.26 per cell. Thus, we have confirmed the capacity to develop monoclonal antibody conjugated niosomes targeted to specific cell receptors, namely CD44.

### EXAMPLE 6

### Materials and Methods

6.1—Materials

Niosome preparations and surfactant derivitizations were made from sorbitan monostearate (Span 60), polyoxyethelene sorbitan monostearate (Tween 61), cholesterol, and dicetyl phosphate (DCP), diisopropylethylamine (DIPEA), cyanuric chloride (CC), and fetal bovine serum (FBS) all came from Sigma Chemical, St Louis, Mo. Fluorescent dyes, 5 (6) carboxyfluorescein (CF) and 5(6) carboxyrhodamine (CR) were obtained from Biotium, Hayward, Calif. Phosphate buffered saline (PBS), borate buffer pH 11.0, Sephadex G50, Dulbecco's Modified Eagle Medium (DMEM), Histochoice tissue fixative, Hank's Balanced Saline (HBS), and goat serum were obtained from Fisher Scientific, Suwannee Ga. Alexa Fluor 488 (AF) came from Molecular Probes, Carlsbad, Calif. IM7 antibodies were provided by Dr K Mikecz of Rush University Medical Center, Chicago, Ill. Collegenase P was obtained from Roche Applied Science, Indianapolis Ind. Bovine synoviocytes where obtained by primary culture described below. Immuno-histochemical staining kit was obtained through Vector Labs, Burlingame, Calif.

6.2—Immunoniosome Synthesis

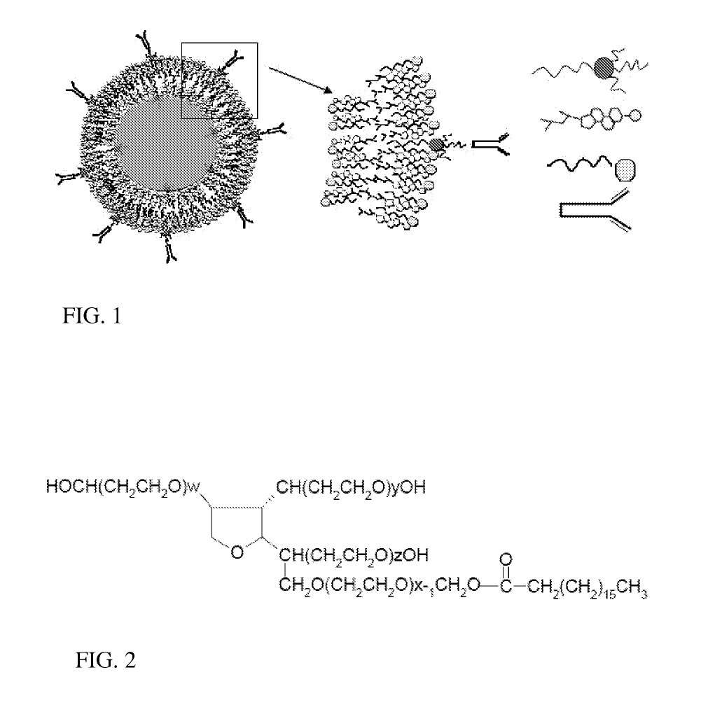

To enable site-specific targeting, niosomal surface modification is needed. The overall scheme includes first chemically modifying a surfactant component, polyethelyne sorbitan monostearate to create a linker, then incorporating the linker within the niosome membrane, and then incubating the functionalized niosomes with monoclonal antibodies to achieve conjugation. The resulting vesicle is modeled in FIG. 1.

6.2.1—Surfactant Derivatization

In order to conjugate a targeting moiety to a niosome, we needed to conceive of a linking agent to be either attached or inserted into the niosomes after they are formed or to be incorporated within the membrane during formation. The last approach would not require an additional process step or potentially affect vesicle stability. Niosomes composed of sorbitan monoesters have been widely studied, with those composed of Span 60 reported as forming the most stable vesicle (Yoshioka et al., International Journal Of Pharmaceutics, 105, 1-6, 1994). The surfactant Tween 61 shown in FIG. 2 is nearly identical in structure to Span 60 except for the additional incorporation of polyethylene branches on the hydrophilic head group. The polyethylene oxide (PEO) groups on the polar head of Tween 61 surfactant potentially could be exploited as a linker for antibody conjugation as has been done with the antibody coupling on the distal end of PEG groups added to immunoliposomes (Allen et al., Biochimica Et Biophysica Acta-Biomembranes, 1237, 99-108, 1995; Bendas et al., International Journal Of Pharmaceutics, 181, 79-93, 1999; Sapra and Allen, Progress In Lipid Research, 42, 439-462, 2003; Torchilin, Journal Of Controlled Release, 73, 137-172, 2001; Zalipsky et al., Liosomes, Pt D, 2004).

Tween 61 was functionalized prior to niosome synthesis by activation the hydroxyl groups on the ends of the polyethylene oxide (PEO) chains. In the presence of diisopropyl ethyl amine (DIPEA), Tween 61 and cyanuric chloride are incubated in a nitrogen environment. The overall mechanism is shown in FIG. 3. The cyanuric chloride undergoes nucleophilic substitution binding to the terminal hydroxyl group of a PEO chain on the Tween 61 molecule. The molar ratio of Tween:CC:DIPEA was 1:0.8:2 (Bendas et al., International Journal Of Pharmaceutics, 181, 79-93, 1999) and a 0.2 g/ml solution was made by combining ig Tween 61, 0.124 g CC, 0.274 ml DIPEA, and 5 ml chloroform. The Tween 61 and chloroform were combined in a round bottom flask. Cyanuric chloride was added and the DIPEA was withdrawn from a sealed flask using a long sharp metal syringe tip and added directly into the mixture. The flask was rotated in a nitrogen environment for 36 hours. The excess solution was stored and remained stable at −4° C. for several months. The resulting functionalized Tween-CC solution added to the surfactants and lipids in chloroform prior to forming a thin film.

6.2.2—Niosome Synthesis

Niosomes were synthesized by thin film hydration techniques using both agitation and bath sonication during the hydration phase. Vesicles are composed of a 1.0:1.0:0.1 molar ratio of surfactant:cholesterol:DCP at a lipid concentration of between 0.0144 to 0.144 M. Optimal molar ratios of vesicle components of sorbitan monoester niosomes have been well described (Yoshioka et al., International Journal Of Pharmaceutics, 105, 1-6, 1994). The lipids were dissolved in chloroform and dried in a 50 ml round bottom flask rotating under a 3 L/min steady stream of N₂ gas to form a thin film on a rotary evaporator (Büchi Rotovapor R200, Brinkmann Instruments, Westbury N.Y.) Once dried, the thin films were hydrated with either 0.01 M PBS, 5.0 mM CF, or 1.0 mM CR rotating for 1-2 hours in a 60° C. water bath. At regular intervals during the hydrations the solutions were agitated on a vortex mixer (Touch Mixer 231, Fisher Scientific, USA). Once complete, the solution was sonicated for 30-60 mins in a bath sonicator (G1125PIG Laboratory Supplies CO, Hicksville, N.Y.) at 80 KHz and 80 watts. Residual chloroform after 24 hours was measured to be less than 0.35% of original solvent by mass before hydration.

Niosomes were separated from unencapsulated dye and unformed lipids by passing the vesicles through a Sephadex G50 column (Superdex HiLoad XK 16/60, GE Healthcare, Piscataway, N.J.) at 1 ml/min with 0.01 M PBS as the elution buffer. Due to the high concentration of the lipids in the 0.144 M niosome preparations, those suspensions are reheated to 60° C. prior to injection into the GEC column which is integrated with a fraction collection and monitoring liquid chromatography instrument (AKTAprime, Amersham Biosciences, GE Healthcare, Piskataway N.J.). Entrapment of dye was assessed in two ways, first by disrupting vesicles and measuring fluorescence of entrapped dye in a fluorescence spectrometer (LS-3B, Perkin Elmer. Boston, Mass.) relative to a standard curve, and secondly, by monitoring the UV absorption of the CF during GEC as shown in FIG. 4. Niosomes were disrupted using a 10% Triton X 100 solution in deionized water at a 1:1 dilution. The final fluorescence intensity was used to obtain the entrapment of dye. Entrapment overall is defined as the amount of dye, in moles, initially added in the formation of niosomes in the hydrating fluid to that recovered after purification multiplied by a dilution factor. Niosomes elute in the void volume (v₀) and free dye in the latter peak.

Formation of vesicles was assessed by light and fluorescent microscopy. Mean particle size and distribution of formed vesicles was determined by light scattering and obscuration techniques (Accusizer 780A, Particle Sizing Systems. Santa Barbara, Calif.).

6.2.3—Antibody Conjugation

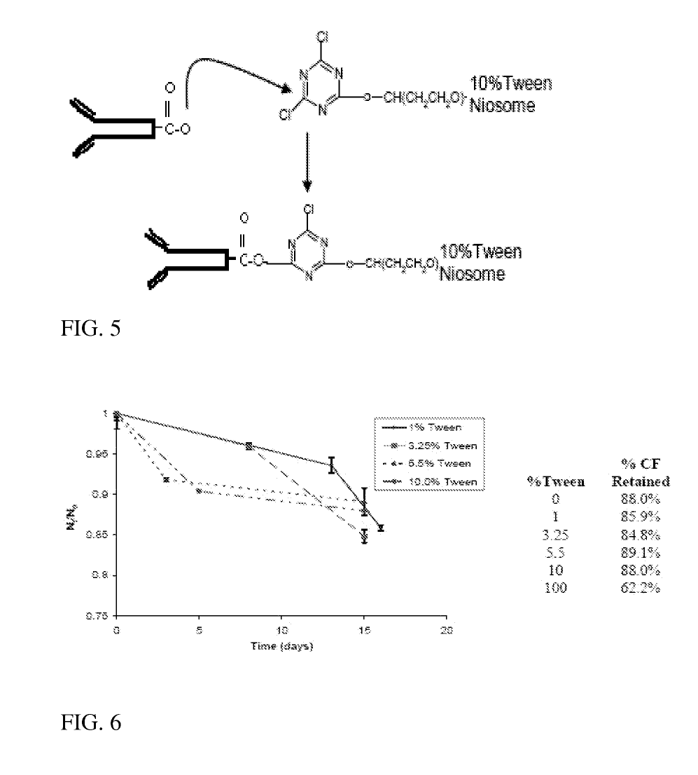

The GEC purified niosome solutions were adjusted to pH 8.8 using borate buffer (Ultrabasic UB10, Denver Instruments, Denver Co.). IgG monoclonal antibodies (either fluorescently tagged AF-IgGs or anti CD44 IM7) were incubated with the niosomes at a concentration of and gently shaken for 16 hours in the dark. At pH 8.8 the binding of a terminal carboxyl group on the antibody is preferred over that of an amino group at the antigen binding terminus shown in FIG. 5. After antibody conjugation the immunoniosome solution pH is restored to 7.4 using 0.1 M PBS. Concentration of antibodies incubated was 5 μg protein/ml niosomes which is equivalent to 2.78 μg protein/μmol lipid. Concentration of total lipids in the post GEC niosome solution is 1.8 mM, found by calculation from the original hydration concentration of 0.0144 M accounting for the 6× dilution factor and particle retention efficiency during GEC. Viable binding groups of Tween-CC linkage are 52% of the Tween component by calculation. This value is based on the reaction molar ratio (1:0.8 of Tween:CC) and a published binding efficiency (65%) of the reaction of CC with 1.2-Dipalmitoyl-sn-glycero-3-phosphoethanolamine polyethylene glycol (DPPE-PEG) (Bendas et al., International Journal Of Pharmaceutics, 181, 79-93, 1999). The overall molar percentage of Tween in the total lipid concentration of the niosomes was 4.76%, making the Tween-CC linker 2.47% of total lipids, or 0.045 μmol TweenCC/ml niosomes. This provides 2.68 * 10¹⁶ binding sites/ml niosomes. Antibody incubation of 5 μg antibodies/ml of niosomes relates to a ratio of greater than 1300:1 Tween-CC binding sites to antibodies.

6.3—Cell Culture

Primary cell cultures of bovine synoviocytes were used in niosome incubation experiments. Synovial membranes were harvested from the metacaralphalengeal joints of 3 month old bovines. After washing with PBS and the tissues were dispersed in 0.1% collagenase P in 4% BSA. Isolated cells were washed with PBS suspended in DMEM containing 10% fetal calf serum at a concentration of 10⁵ cells per ml. The cells were plated in Labtek 8 well microslides. Cells were allowed to attach overnight. The media was removed, and the cells washed with PBS and fixed for 2 hours in Histochoice. Select fixed cells were immuno-stained with IM7 using standard immunohistochemical techniques.

6.4—Experimental Methods

6.4.1 Niosome Antibody Conjugation

Prior to immunoniosome-cell binding experiments, the coupling of the Tween-CC linker incorporated within the niosome to an antibody was tested by conjugating fluorescently tagged Alexa-488 (AF) rat IgGs to non-fluorescing (PBS containing) niosomes. Structurally similar to CF, AF absorbs UV at 280 nm and the monitoring of absorbance during GEC can be used to verify the presence of the AF tagged antibodies conjugated to non fluorescing niosomes during the elution profile. To further verify the antibody binding to the niosomes, the post GEC purified AF-immunoniosomes were examined with an Olympus 1X71 inverted fluorescent microscope. Approximately 50 μl of immunoniosome suspension was pipetted onto a glass microscope slide and viewed at 10 and 40× using a FITC filter to verify the presence of fluorescent spherical particles. Fluorescent images where captured using DP-BSW software.

6.4.2—Immunoniosome Fixed Cell Binding

Once coupling was verified, binding of immunoniosomes to target antigen in a fixed cell model was assessed. The fixed cell layers were pre-incubated in 0.01 M PBS with 2% goat serum with or without soluble IM7 antibody for 1 hour at room temperature prior to incubation with immunoniosomes. Cells were rinsed with PBS and incubated for 1 hour at 37° C. with fluorescent niosomes or fluorescent niosomes derivatized with IM7. The cells were well rinsed to remove unbound niosomes and examined by fluorescent microscopy. For a given image, a phase contrast picture was captured, and then the light and filters were changed to fluorescent mode to image the cell nuclei using the DAPI filter, and the FITC filter to image the fluorescent niosomes bound to the cells. These images were captured and combined using DP-BWR image analysis software to overlay them.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A niosome for targeted delivery of a bioactive agent comprising
a niosomal membrane comprising polyoxyethylene sorbitan monostearate;
an antibody or fragment thereof having specific affinity for a cell surface antigen wherein the antibody is covalently coupled to the polyoxyethylene sorbitan monostearate.

2. The niosome according to claim 1 wherein the antibody or fragment thereof is an anti-CD44 antibody.

3. The niosome according to claim 1 further comprising a bioactive agent.

4. The niosome according to claim 3 wherein the bioactive agent is an anti-inflammatory agent.

5. The niosome according to claim 1 wherein the niosomal membrane further comprises sorbitan monostearate.

6. The niosome according to claim 5 wherein the molar ratio of polyoxyethylene sorbitan monostearate to sorbitan monostearate is about 1:5 to about 1:20.

7. The niosome according to claim 5 wherein the molar ratio of polyoxyethylene sorbitan monostearate to sorbitan monostearate is about 1:10.

8. A delivery reagent for the targeted delivery of a bioactive agent comprising:
a niosomal membrane comprising a nonionic surfactant molecule;
an antibody or fragment thereof having specific affinity for an antigen; and
a cyanuric chloride linker wherein the cyanuric chloride linker couples the antibody or fragment thereof to the nonionic surfactant molecule.

9. The delivery reagent according to claim 8 wherein the antibody or fragment thereof is an anti-CD44 antibody.

10. The delivery reagent according to claim 8 further comprising a bioactive agent.

11. The delivery reagent according to claim 8 wherein the bioactive agent is an anti-inflammatory agent.

12. The delivery reagent according to claim 8 wherein the nonionic surfactant molecule is polyoxyethylene sorbitan monostearate.

13. The delivery reagent according to claim 12 wherein the niosomal membrane further comprises sorbitan monostearate.

14. The niosome according to claim 13 wherein the molar ratio of polyoxyethylene sorbitan monostearate to sorbitan monostearate is about 1:5 to about 1:20.

15. The niosome according to claim 13 wherein the molar ratio of polyoxyethylene sorbitan monostearate to sorbitan monostearate is about 1:10.

16. A niosome for targeted delivery of an agent comprising
a niosomal membrane comprising polyoxyethylene sorbitan monostearate;
an antibody or fragment thereof having specific affinity for a cell surface antigen wherein the antibody is covalently coupled to the polyoxyethylene sorbitan monostearate.

17. A method of preparing an immuno-conjugated niosome comprising the steps of:
providing a nonionic surfactant molecule comprising a terminal hydroxyl group on a chain of the molecule;
functionalizing the hydroxyl group by addition of cyanuric chloride;
constituting niosomal vesicles comprising the functionalized nonionic surfactant molecule; and
reacting the functionalized nonionic surfactant molecule with an antibody or fragment thereof to form a nonionic surfactant molecule covalently coupled to an antibody.

18. The method according to 17 wherein the constituted niosomal vesicle comprises a bioactive agent.

19. The method according to claim 17 wherein the nonionic surfactant molecule comprising a terminal hydroxyl group is polyoxyethylene sorbitan monostearate.

20. The method according to claim 19 wherein the constituted niosomal vesicle comprises sorbitan monostearate.

21. The method according to claim 20 wherein the molar ratio of the functionalized nonionic surfactant molecule to sorbitan monostearate is about 1:5 to about 1:20.

22. The method according to claim 20 wherein the molar ratio of the functionalized nonionic surfactant molecule to sorbitan monostearate is about 1:10.
